(19) United States
(12) Patent Application Publication
(10) Pub. No.: US 2007/0238995 A1
(43) Pub. Date: Oct. 11, 2007

(54) Medical diagnostic ultrasound system with peripheral sensors or data

(76) Inventors: Lei Sui, Newcastle, WA (US); James B. Seward, Rochester, MN (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/373,789

(22) Filed: Mar. 10, 2006

(51) Int. Cl. A61B 8/00

(52) U.S. Cl. 600/437

(57) ABSTRACT

Medical diagnostic ultrasound integrates with peripheral sensors or data. An ultrasound system includes connectors or inputs for receiving information from other types of sensors, such as blood pressure, tonometry, central aortic pressure, blood sugar, cholesterol, or other vascular characteristic sensors. An algorithm, such as stored on a computer readable media as instructions, uses the ultrasound data and data from the other type of sensor, such as pressure data obtained from a limb, to determine a risk or diagnosis. In one method, a peripheral vascular measurement of central aortic pressure, such as obtained by using a tonometer on a patient's wrist, is used with ultrasound data to determine a risk or diagnosis and/or automatic data reading/cross-checking.

### BACKGROUND

The present embodiments relate to medical diagnostic ultrasound. In particular, cardiovascular function is assessed with medical diagnostic ultrasound and peripheral sensors or data.

Cardiovascular disease (CVD) is one of the most common diseases. CVD may be deadly, but is also treatable. Early warning of a risk of CVD may more likely lead to successful treatment. To determine risk, various studies have tracked different factors. One study is the Framingham Heart Study for risk stratification. Ultrasound information has been used in the ongoing protocol of the Framingham Study. Another study is the Israeli Ischemic Heart Disease Project. Yet another study is the Cardiovascular Health Study. Common risk predictors from these studies include age, blood pressure (BP), cholesterol level, diabetes, smoking history, CVD history, atrial fibrillation, and left ventricular hypertrophy (LVH).

For calculating risk, data is acquired utilizing a single acquisition device (i.e., ultrasound, CT, etc.). Other data may include general patient information. The data is collated based on an individual physician's expertise or general understanding. Such information is often not generally available or uniformly applied within the medical community. There is a tendency to experience inconsistencies between various users.

### BRIEF SUMMARY

By way of introduction, the preferred embodiments described below include methods, systems and computer readable media for medical diagnostic ultrasound with peripheral sensors or data. An ultrasound system includes connectors or inputs for receiving information from other types of sensors, such as blood pressure, tonometry, central aortic pressure, blood sugar, cholesterol, or other vascular characteristic sensors. An algorithm, such as stored on a computer readable media as instructions, uses the ultrasound data and data from the other type of sensor, such as pressure data obtained from a limb, to determine a risk or diagnosis and to cross check data validity. In one method, a peripheral vascular measurement of central aortic pressure, such as obtained by using a tonometer on a patient's wrist, is used with ultrasound data to determine a risk or diagnosis.

In a first aspect, a medical diagnostic ultrasound system is provided for assisting in diagnosis. The system includes an ultrasound data path for scanning with ultrasound energy and generating ultrasound data as a function of the scanning. A peripheral connector is operable to connect with a peripheral vascular characteristic sensor. The peripheral connector is operable to receive vascular data from the peripheral vascular characteristic sensor. A processor connects with the ultrasound data path and the peripheral connector such that the processor is operable on the ultrasound data and vascular data.

In a second aspect, a medical diagnostic ultrasound system is provided for assisting in diagnosis and/or in data reading. An enclosure has an input connectable with a pressure sensor. An ultrasound imaging system connects with the enclosure. A processor connects with the enclosure. The processor is operable to process data from the ultrasound imaging system and pressure data received at the input.

In a third aspect, a method is provided for medical diagnostic ultrasound. Ultrasound data is acquired with an ultrasound imaging system. A peripheral vascular measurement of central aortic pressure is acquired. A processor determines a risk. data validity or diagnosis as a function of both the ultrasound data and the peripheral vascular measurement.

In a fourth aspect, a computer readable storage medium has stored therein data representing instructions executable by a programmed processor for medical diagnostic ultrasound. The instructions are for receiving ultrasound data and pressure data, determining a risk or diagnosis as a function of both the ultrasound data and the peripheral vascular measurement, and outputting the risk or diagnosis, or crosschecking data validity.

The present invention is defined by the following claims, and nothing in this section should be taken as a limitation on those claims. Further aspects and advantages of the invention are discussed below in conjunction with the preferred embodiments and may later be claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of a medical diagnostic ultrasound system and peripheral components in one embodiment;

FIG. 2 is a flow chart diagram of one embodiment of a method for assisting in diagnosis; and

FIG. 3 is a graphical representation of vascular pressure data.

### DETAILED DESCRIPTION OF THE DRAWINGS AND PRESENTLY PREFERRED EMBODIMENTS

Complementary technologies, such as ultrasound and pressure tonometry, classify risk of cardiovascular or other disease. By using an integrated system, data for the different technologies is automatically obtained or solicited. In one embodiment, the cardiovascular system is classified separately for reservoir (e.g., atria and/or veins), pump (e.g., ventricle), and tube (e.g., arteries, arterioles and/or capillaries) components. The entire cardiovascular system is accounted for rather than only the pump and/or reservoir. Different combinations of the complementary technologies are used to classify the different components, such as ultrasound for reservoir and pump and pressure tonometry for tube. A knowledge-based system incorporates various probabilities or other diagnostic information to classify based on data acquired for a particular patient. Wellness or disease burden is expressed using an algorithm. An environment of artificial intelligence, such as models, neural network, filters, matrices or other classifiers, communicate the output information, data inconsistencies, and/or a suggested course of action.

Risk stratification and/or automatic data reading are provided by integrated data acquisition devices operated pursuant to algorithm driven acquisition. The acquired data is arranged or analyzed based on a knowledge base implemented to create knowledge, suggest solutions, indicate probabilities and/or output risk. The integrated device to acquire and process the data may avoid having to perform different tests at different times. Integrating ultrasound and other non-invasive sensors, such as tonometry, allows convenient diagnosis, reducing data reading time and improving quality. An output combined from different modalities may provide assessment that is more comprehensive to the physician or patient.

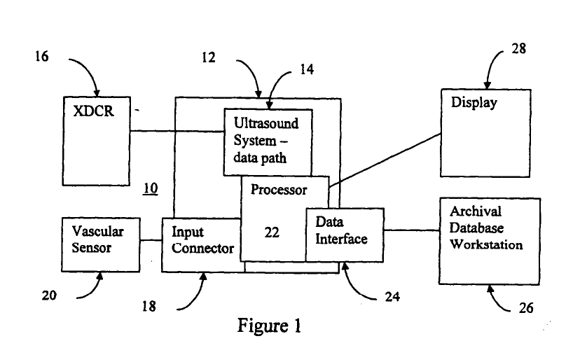

FIG. 1 shows a system 10 for assisting in diagnosis. The diagnosis is for cardiovascular disease, but may be for other diseases. The assistance is to provide risk, diagnosis or other information by classifying input or acquired information.

The system 10 includes an enclosure 12, a medical diagnostic ultrasound system 14, an input connector 18, a data interface 24, a processor 22 and a display 28. The input connector 18 allows connection of a vascular sensor 20 to the enclosure 12, and the data interface 24 allows input or export of data, such as to or from the archival database or workstation 26. Additional, different or fewer components may be provided, such as providing for EKG input.

The enclosure 12 completely encloses the ultrasound system 14 and the processor 22, except for one or more ports for a transducer connector, vascular sensor 20, data interface 24, input device connections and display output. Alternatively, the enclosure 12 includes doors, openings or otherwise incompletely encloses one or more components. The enclosure 12 is one or more housings, such as provided on or being a medical diagnostic ultrasound system. Such enclosures 12 may be cart mounted, handheld, or portable. For example, a cart mounted system 10 includes a same or separate housings for different components all connected as a same or one device, a cart mounted diagnosis assistance and data acquisition system. Electrical connections between the components may be routed by cables, data bus, or other conductors external to or within the housings. The ultrasound system 14, input connector 18, processor 22 and data interface 24 connect with the enclosure physically (e.g., brackets or other mountings) and/or electronically.

The input connector 18 is an input (e.g., port, opening, cord or plug) on the enclosure 12. The input connector 18 is a USB, serial, parallel, mouse, keyboard, audio, microphone, Ethernet, firewire, wireless, custom, combinations thereof or any other now known or later developed connector for physical and/or electrical connection. The input connector 18 allows for detachable or permanent connection. The input connector 18 provides connection with a peripheral. The peripheral connector is operable with a specific type or group of peripheral sensors 20. Alternatively, the peripheral connector is operable with any type of sensor having a mating connector. The peripheral connector may allow for output of data or control to the sensor 20.

The peripheral sensor 20 is peripheral to the ultrasound system 14 and/or the enclosure 12, such as being on a cable extending from the enclosure 12. Alternatively or additionally, the peripheral sensor 20 is a sensor for sensing a characteristic from the periphery of a patient (e.g., the arm). The peripheral connector receives raw or processed information from the peripheral sensor when connected.

In one embodiment, the peripheral sensor 20 is a vascular characteristic sensor for measuring a characteristic of a patient's vascular system. For example, the peripheral sensor 20 is a pressure sensor. One type of pressure sensor is a blood pressure sensor. A cuff, pump and circuitry are provided for determining the blood pressure from a patients arm, such as the brachial blood pressure. Another type of pressure sensor is a tonometer. In tonometry, a patient's radial artery pressure is measured. Automated or manual placement of the sensor 20 is used. The radial artery pressure may be used by the sensor 20 or the processor 22 to determine or estimate a central aortic pressure or associated characteristic. Other types of sensors 20 include a blood sugar sensor or a cholesterol sensor. Using infrared or ultraviolet measurements or blood sampling (e.g., a prick), the blood sugar and/or cholesterol may be measured. Other now known or later developed vascular characteristic sensors may be used. By integrating one or more sensors 20, a more complete characterization of the vascular system of a patient may be obtained. For example, arterial tonometry integrated with the system 10 may allow for characterization of the arteries, arterioles and/or capillaries.

The ultrasound system 14 includes an ultrasound data path for scanning with ultrasound energy and generating ultrasound data as a function of the scanning. For example, the transducer 16 or a transducer connector operates with transmit and receive beamformers. The output of the receive beamformer is provided on a B-mode path, a color Doppler path and/or a spectral Doppler path. Detectors detect the intensity, velocity, energy, velocity range, turbulence or other characteristic for a location, two-dimensional area or a three dimensional volume. An optional scan converter formats the detected information as an image for the display 28. Additional, different or fewer components may be provided, such as including filters and/or image processors. Other ultrasound imaging systems may be used.

The display 28 is a CRT, LCD, plasma, projector, printer or other now known or later developed display device. The display 28 connects with the processor 22, but may alternatively connect to the ultrasound system 14 or through the data interface 24. The display 28 is part of, rests on, adjacent to or remote from the enclosure 12.

The data interface 24 is an Ethernet, firewire, modem, user interface (e.g., keyboard or other input in combination with the display 28), network connector, wired or wireless, combinations thereof, or other now known or later developed device for exchanging data. In one embodiment, the data interface 24 connects with a workstation or archival database 26, such as a PACs system. In another embodiment, the data interface 24 accepts manual input of information from a user. The data interface 24 is operable to receive patient information, such as previously acquired images (e.g., ultrasound, x-ray, computed tomography, magnetic resonance, positron emission, or others). Other patient information includes age, weight, gender, whether diabetic, test results (e.g., blood sugar or cholesterol from a blood test), diagnoses, combinations thereof, or other information acquired for a patient. The data interface 24 automatically obtains all or a sub-set of patient information based on a user's indication of a particular patient. Alternatively, the data interface 24 allows navigation by the user to acquire the desired information. The user may be prompted to acquire particular types of data to be used for classification.

The processor 22 is a general processor, control processor, digital signal processor, application specific integrated circuit, field programmable gate array, digital circuit, analog circuit, combinations thereof, or other now known or later developed device operable to process information. The processor 22 connects with the enclosure 12, such as being within the enclosure 12. In one embodiment, the processor 22 is a processor of the ultrasound system 14, but may be separate. In another embodiment, the processor 22 is remote from the enclosure, such as being part of the workstation 26.

The processor 22 connects with the ultrasound system 14, the input connector 18 and the data interface 24 for data communication. Direct or indirect connections may be used. The processor 22 solicits the data or receives the data without triggering. The processor 22 controls the acquisition of data, such as ultrasound, pressure, blood pressure, blood sugar, cholesterol or other information. The information is acquired through the ultrasound system 14, the input connector 18, and/or the data interface 24. To acquire the data, the processor 22 automatically performs measurements or causes operation of the controlled components. Alternatively or additionally, the processor 22 prompts action or measurements by the user, such as instructing the user to perform specific ultrasound scans (e.g., A4C, A2C, or pasternal views) and to perform a tonometer measurement. The user may indicate which data or information should be recorded, such as once the desired view is obtained. Providing control through the processor 22 of the data acquisition from multiple devices may improve workflow and increase the likelihood of obtaining proper data for classification. Integration of some or all of the multiple devices with or without access to archived or networked information from other sources may likewise assist in obtaining proper data for classification or risk stratification.

The processor 22 is operable to process data from the ultrasound imaging system 14, data from the data interface 24, and/or pressure or vascular data received at the input connector 20. In one embodiment, the processor 22 controls export of the data, such as transmitting the data through the data interface 24. Classification is performed remote from the system 10 or enclosure 12, such as in the workstation 26 in an offline mode. In online embodiments, the processor 22 performs filtering, calculations, data extraction from images, classification, report generation, or other processing functions using some or all of the acquired data. Any results are displayed on the display 28 or transferred through the data interface 24.

The processor 22 determines a risk or diagnosis, or data validity as a function of the data, such as ultrasound and vascular data. Any now known or later developed model, neural network or other classification algorithm may be used. The determination may be a function of a knowledge base, such as specific parameters known to have probabilistic indications of risk. More than one risk determination may be made. For example, risk is determined from two or more models. As another example, different risks (e.g., current health and five year risk of cardiac event) are calculated. Risk associated with different portions of the vascular system may be calculated, such as determining a health status of the left ventricle, left atrium and aortic arteries. The risk or diagnosis is output to the display 28 or through the data interface 24.

Data validity is determined by cross checking the data. The data check may be between two types of data, such as between tonometry data and ultrasound data. For example, tonometer measurements may indicate good health, but one or more ultrasound measurements may indicate a high risk. The inconsistency is output as a data validity measure or flag. The user may perform the same or different tests or measurements to attempt to resolve the inconsistency. Where there are no inconsistencies, the validity of the data may be indicated to the user. As another example, the vessel thickness is measured using ultrasound (e.g., IMT) to validity tonometer readings.

The system 10 includes a memory, such as memory for the processor 22, in the ultrasound system 14, or on a removable media. The memory is a computer readable storage medium having stored therein data representing instructions executable by a programmed processor, such as the processor 22, for medical diagnostic ultrasound.

The instructions implement the processes, methods and/or techniques discussed herein. The computer readable media may be a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, filmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

In one embodiment, the instructions control the processor 22. The instructions are for receiving ultrasound data, pressure data, other vascular data and/or other data. For example, the instructions are for the processor 22 to receive a tonometer measurement, such as a central aortic pressure. The instructions may implement an automated or semiautomatic workflow using a single user interface to acquire ultrasound data, the peripheral vascular measurement and/or other data.

The instructions may cause the processor 22 to process the acquired data to determine one or more parameters. For example, the processor 22 determines a ventricle volume, ventricle ejection fraction, wall velocity, atria volume, valve velocity, flow velocity, deceration time or other measure of the heart from an image, such as an ultrasound image. Any now known or later developed quantification package for cardiovascular imaging may be used, such as the automated or semi automated algorithm provided with Siemens or other manufacturer cardiovascular ultrasound systems.

The instructions cause the processor 22 to determine a risk, diagnosis or data validity as a function of the acquired data, such as ultrasound data and the peripheral vascular measurement. By comparing the peripheral vascular measurement and the ultrasound data to a knowledge base, the health or risk of the cardiovascular system may be classified. Other classification approaches may be used with or without a knowledge base. The overall cardiovascular system or specific portions are classified. For example, the operation of atria or veins and ventricle is characterized as a function of the ultrasound data, and the operation of arteries, arterioles or capillaries is characterized as a function of the pressure data. Other data may also be used in one or more of the classifications.

The instructions cause the processor 22 to output the risk or diagnosis information to the user. The output is textual, numerical, graphical or combinations thereof. The underlying information, such as image-extracted measurements, patient information, and vascular measurements may also be output.

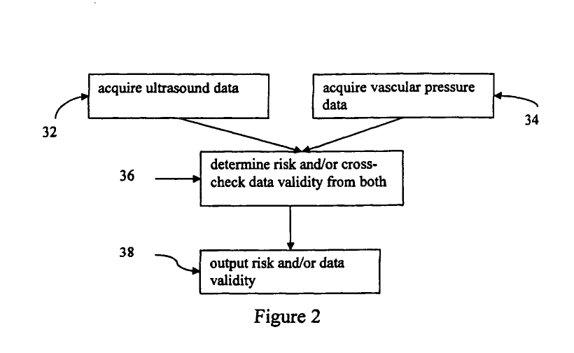

FIG. 2 shows a method for classification using medical diagnostic ultrasound. The method of FIG. 2 is implemented by the system 10 of FIG. 1 or a different system. The acts shown in FIG. 2 are performed in the order shown or a different order. Additional, different or fewer acts may be provided.

In act 32, ultrasound data is acquired. The ultrasound data is acquired from a memory, such as an archival system. Alternatively, the ultrasound data is acquired in real-time with the classification, just prior to classification or as part of the classification process using an ultrasound imaging system.

The ultrasound data represents an image or parameters extracted from an image. In one embodiment, the image data represents apical four chamber (A4C), apical two chamber (A2C), and short axis (SAX) views. Different views may be acquired. B-mode or intensity data and Doppler tissue or tissue velocity data is acquired in one or more, such as all of the views. Doppler flow information may be acquired. Spectral or pulsed wave (PW) Doppler information for one or more points may be acquired. M-mode data may be acquired.

Parameters are extracted from the images or ultrasound data. For example, a left ventricle ejection fraction (LVEF), left atria volume (LAV), left ventricle mass (LVM), maximum velocity of the myocardium (E′), mitral valve flow velocity (E), mitral valve deceleration time (DT), peak systolic Doppler tissue imaging (DTI) velocity (Sm), and a ratio of the mitral valve maximum velocity to the velocity of at the A wave (E/A) are extracted. Left ventricle mass may be determined from the SAX view. Left ventricle volume, LVEF, and atrial volumes may be determined from the A4C and A2C views. Mitral Doppler information in the A4C view may be used to determine E, A and PHT for deriving the deceleration time. The PW tissue Doppler information with a gate placed in the A4C view may be used to derive Ea, Aa and Sa. Some information, such as imaging (e.g., PW DTI) or parameters (e.g., LVM and LAV) may not be acquired. The parameters are extracted using any now known or later developed algorithm. Alternatively, the parameters are input by the user based on the acquired image information.

In act 34, a peripheral vascular measurement is acquired. A peripheral sensor connected with an ultrasound imaging system or a separate sensor performs the vascular measurement. For example, a tonometer connected with the ultrasound imaging system measures pressure. Other sensors may be used. The vascular measurement is automated, semi automated or manual. One or more parameters are measured one or more times. For example, blood sugar, blood pressure, aortic central pressure, cholesterol, or combinations thereof are measured one or more times. As another example, one minute or multiple heart cycles of radio tonometry information is acquired. The vascular measurement is the raw data or information derived from the raw data.

Acts 32 and 34 are performed sequentially or in parallel. The acts 32 and 34 are performed as separate tests due to physician's requests. Alternatively, acts 32 and 34 are performed in an automated or semi automated workflow of a single user interface. Using a multifunctional device or portfolio of devices may provide all or much of the information for a particular or predetermined need, such as cardiovascular assessment. The data acquired by the devices is collated into “knowledge” to take advantage of a more broad based understanding. Technology and information over a broad range of devices or fields may be useful in diagnosis. Acquisition is automated or driven by a focused algorithm. Based on the level of knowledge imparted, the user's knowledge may be elevated to a higher level of understanding.

In act 36, a processor determines a risk or diagnosis as a function of the ultrasound data, the peripheral vascular measurement, and/or other information. A device in the ultrasound imaging system or a separate device determines the risk or diagnosis. Ultrasound data plus other complementary data may provide specific information for the major components of cardiovascular system. Using a processor and integrated data gathering devices may reduce data reading time, improve reading quality, and makes it easier to determine and transfer health information. Knowledge from studies or physicians and/or training data sets allows the creation of intelligence as a neural network, matrix, algorithm, classifier or knowledge base to discern errors, communicate association, enhance understanding, advance learning and improve the delivery of improved healthcare.

In one embodiment, the cardiovascular risk is determined for different portions of the cardiovascular system. For example, operation of (1) atria or veins, (2) ventricle, and (3) arteries, arterioles or capillaries is characterized. Each portion is characterized as a function of the ultrasound data, the peripheral vascular measurement, other data or combinations thereof. The functional components of the cardiovascular system can be modeled as a series of atria and veins (reservoir), ventricle (pump), and arteries, arterioles and capillaries (tubes). Anatomical and/or functional changes in these components reflect the cumulative burden of known or unknown disease states. Knowledge based risk is a synthesis of pertinent normal and abnormal data derived from the pump, tube and reservoir. This knowledge based risk stratification uses a noninvasive ultrasound device complemented with additional noninvasive hardware and software capable of defining the anatomy and function of each of the three components of the cardiovascular system. The ultrasound device generates images and physiological information using various manipulations of the ultrasound signal (e.g., time-of-flight, Doppler, speckle tracking, or tissue characterization). The insonated tissues can be defined by various anatomical and functional characteristics. Additional devices, such as pressure tonometry, electrical impedance, microvascular flow, wave analysis, biomarker characterization, or others, complement the accrual of pertinent information used to compute knowledge-based risk.

The system includes database storage and retrieval, interactive review and complementary hardware and software. The system is integrated via network solutions and algorithms. The software includes interactive and automated risk algorithms, user interfaces, reports, program interfaces, image and trace processing algorithms, and other supporting solutions. Knowledge based risk is derived from the acquired parameters received and integrated from the various acquisition devices. For knowledge base classification, the peripheral vascular measurements and the ultrasound data or parameters are compared to a knowledge base. The comparison-indicates risk. The fully integrated system extracts characteristic indices, which are collated into knowledge. The supporting programs include other peripherals such as printing or recording, or information presentation. The knowledge-based apparatus may characterize general or specific diseases and functions. The knowledge-based system utilizes artificial intelligence, algorithms, models, filters, matrices or other now know or later developed classifiers to communicate potential errors or inconsistencies in the acquired data set.

In one embodiment, one or more of the risk scoring algorithms or models disclosed in Tsang, et al, “Prediction of Risk for First Age-Related Cardiovascular Events in an Elderly Population: The Incremental Value of Echocardiography,” J. of Am. College of Cardiology, Vol. 42, No. 7, pages 1999-1205 (2003) are implemented as the classifier. Various inputs are used to estimate the risk or probability of a cardiac event in a particular time period, such as in a 5 year time period. The same or different model is used to rank a current cardiac health, such as providing a low, moderate and high risk ranking or providing a score of 1-10 where 1 is the least and 10 is the highest risk or health. Table 1 below shows a correlation of a calculated score 1-10 to a three level risk of a cardiac event in five years.

TABLE 1

| | Score | | |
|---|---|---|---|
| | <=2 | 3-4 | >=5 |
| Risk | Low | Medium | High |
| Prob. | 10% | 26% | 50% |

The score, the risk, the probability or combinations of two or all of the values are output.

In one embodiment, the score is calculated by assigning a numerical value to different risk factors. The total of the numerical values is the output score. Table 2 below shows one example embodiment of numerical values and risk factors.

TABLE 2

Score

| | 0 | 1 | 2 | 3 | 4 |
|---|---|---|---|---|---|
| Age | 65-69 | 70-74 | 75-79 | 80-84 | 85 |
| Gender | Female | Male | | | |
| Diabetes | No | Yes | | | |
| SBP | <140 | >=140 | | | |
| EF | >=50% | | <50% | | |
| Diastolic | No | Yes | | | |
| Problem | | | | | |
| LVMI | <120 g/m | ≧120 g/m | | | |
| LAVI | else | ≧32 cc/m2 | | | |
| | | E′ <= 11 | | | |

In one embodiment, the risk or health is calculated based on the greatest risk factor for a patient. Table 3 below shows one example embodiment for reservoir and ventricular function.

TABLE 3

| | | Vol. | | | |
|---|---|---|---|---|---|
| Reservoir | Normal | Overload | Mild | Mod. | High |
| LAVI | <28 | >=28, | 28-33 | 34-39 | >=40 |
| (cc/m2) | | E′ >= 12; | | | |
| | | 11-10, | | | |
| | | Transient | | | |
| Ventricular | Unit | Normal | Mild | Moderate | High |
| Function | | | | | |
| EF | % | >50 | 50-40 | 39-20 | <20 |
| (Contractility) | | | | | |
| E′ (Relaxation) | cm/s | >=12 | 11 to 8 | 7 to 5 | <5 |
| E/E′ (Filling | | <8 | 8-10 | 10-15 | >=15 |
| Pressure) | | | | | |
| Sa (Systolic | cm/s | >5 | <=5 | | |
| Fx) | | | (abnormal) | | |

If left atrial volume is bigger than 28 cc/m2, usually it infers an abnormality.

However, in some athletes, their hearts are so strong that the volume is enlarged a bit to provide more blood. This case is called volume overload. A diseased atrium is called pressure overload. For example, a LAVI of 35 indicates a moderate risk. As another example, an EF of 19 shows high risk and an E′ of 10 shows low risk. The output for ventricular function would be high risk. The output for reservoir would be moderate risk. An output for cardiac risk based on ventricular function and reservoir would be a high risk.

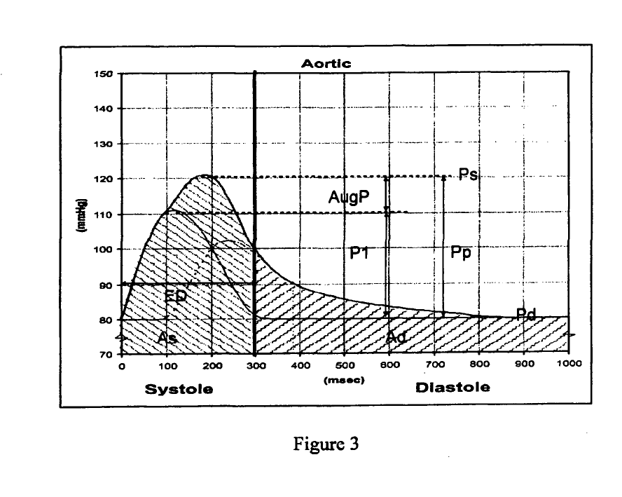

In another embodiment, the health or risk for tubes is determined to provide an overall cardiovascular health or risk indication. For example, the central aortic pressure measured with a tonometer and other values determined with the tonometer data are used to classify risk or health. Table 4 shows one example for classifying between normal and abnormal. FIG. 3 shows the relationship of some of the parameters. Any one parameter in the abnormal range results in an abnormal output for the tubes classification or an increase in an overall risk, such as increasing a general characterization (e.g., moderate to high) or increasing a score value (e.g., from 3 to 5).

TABLE 4

| Tubes | Unit | Normal | Abnormal |
|---|---|---|---|
| Central | mmHg | <=130 | >130 |
| Systolic | | | |
| Blood | | | |
| Pressure | | | |
| Central Pulse | mmHg | <=50 | >50 |
| Pressure | | | |
| Augmentation | | <21 (men) | >=21 (men) |
| Index | | <30 (women) | >=30 (women) |
| Systolic | | 30-40% | <30% |
| Ejection | | | |
| Period | | | |
| Coronary | | >=120% | <120% |
| Perfusion | | | |
| Buckberg | | | |
| Index | | | |

Any combination of risk, scores or probabilities may be used, such as selecting one or weighted combination of two or more. For example, the score and the risk are calculated separately. The separate calculations may be displayed together or combined into a single output. The probability may be based on the score, risk or a separate classification. In one embodiment, the probability is a percentage associated with the three levels of risk. The percentage is determined from a study or training data. In one embodiment, the probability is for a cardiac event within 5 years, but may be a different probability associated with cardiovascular function or for a different time period.

As used herein, the score and probability values indicate risk, but in a different way. The risk may be expressed as textual (high, moderate, low), numerically (1-10), as a percentage chance of an event (probability) or in another way. The health or risk associated with a fewer or greater numbers of portions of the cardiovascular system may be used. Different parameters or threshold values for any one or more of the parameters in the tables above may be used. Other now known or later developed models or other classifiers may be used.

In act 38, the risk or diagnosis is output. For example, a red, yellow or green button or light is output to indicate high, moderate or low risk. As another example, text provides the risk level, such as “Risk: Moderate,” “Risk Score=5,” or “probability of cardiovascular event in 5 years: 25%.” As another example, a graph indicates probability of a cardiac event as a function of time. The graph is based on statistics given the various risk factors or may be a linear graph of probability as a function of time from 0% to a determined 5 year probability. Other representations of the same or different risks may be used.

Other information may be output as well, such as a graphic of a heart with shading or colors indicating risk associated with different locations. The factors considered for any particular risk may be output. The supporting images or other sensor data may be output. For example, FIG. 3 shows an output of tonometer measurements.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I claim:

1. A medical diagnostic ultrasound system for assisting in diagnosis, the medical diagnostic ultrasound system comprising:
an ultrasound data path for scanning with ultrasound energy and generating ultrasound data as a function of the scanning;
a peripheral connector operable to connect with a peripheral vascular characteristic sensor, the peripheral connector operable to receive vascular data from the peripheral vascular characteristic sensor; and
a processor connected with the ultrasound data path and the peripheral connector such that the processor is operable on the ultrasound data and vascular data.

2. The medical diagnostic ultrasound system of claim 1 wherein the peripheral connector comprises a tonometer connector operable to connect with a tonometer, and wherein the processor is operable to process pressure information.

3. The medical diagnostic ultrasound system of claim 2 wherein the tonometer connector comprises a central aortic pressure sensor.

4. The medical diagnostic ultrasound system of claim 1 wherein the peripheral connector comprises a blood pressure sensor connector, and wherein the processor is operable to process blood pressure information.

5. The medical diagnostic ultrasound system of claim 1 wherein the peripheral connector comprises a blood sugar sensor connector, and wherein the processor is operable to process blood sugar information.

6. The medical diagnostic ultrasound system of claim 1 wherein the peripheral connector comprises a cholesterol sensor connector, and wherein the processor is operable to process cholesterol information.

7. The medical diagnostic ultrasound system of claim 1 wherein the ultrasound data path comprises a B-mode path and a Doppler path;
further comprising a display, the processor operable to output data to the display as a function of the ultrasound data and the vascular data.

8. The medical diagnostic ultrasound system of claim 1 wherein the ultrasound data path, the peripheral connector and the processor are in a same device, and wherein the processor is operable to determine a risk or diagnosis as a function of both the ultrasound data and the vascular data.

9. The medical diagnostic ultrasound system of claim 1 further comprising:
a data interface operable to receive patient information of age, weight, gender, whether diabetic, or combinations thereof;
wherein the processor connects with the data interface such that the processor is operable on the patient information.

10. The medical diagnostic ultrasound system of claim 1 wherein the processor is operable on the ultrasound data and vascular data by being operable to transmit the ultrasound data and vascular data from the medical diagnostic ultrasound system.

11. A medical diagnostic ultrasound system for assisting in diagnosis or data reading, the medical diagnostic ultrasound system comprising:
an enclosure having an input connectable with a pressure sensor;
an ultrasound imaging system connected with the enclosure;
a processor connected with the enclosure, the processor operable to process data from the ultrasound imaging system and pressure data received at the input.

12. The medical diagnostic ultrasound system of claim 11 wherein the input comprises a tonometer input.

13. The medical diagnostic ultrasound system of claim 11 wherein the input comprises a central aortic pressure sensor input.

14. The medical diagnostic ultrasound system of claim 11 wherein the input comprises a blood pressure sensor connector.

15. The medical diagnostic ultrasound system of claim 11 wherein the ultrasound imaging system comprises a B-mode path and a Doppler path, and wherein the enclosure has a data interface operable to receive patient information of age, weight, gender, whether diabetic, or combinations thereof;
further comprising:
a display connected with the processor and the ultrasound imaging system
wherein the processor is operable to determine a risk or diagnosis as a function of the ultrasound data, the patient information, and the pressure data and is operable to output the risk or diagnosis to the display.

16. A method for medical diagnostic ultrasound, the method comprising:
acquiring ultrasound data with an ultrasound imaging system;
acquiring a peripheral vascular measurement of central aortic pressure; and
determining, with a processor, a risk, data validity or diagnosis as a function of both the ultrasound data and the peripheral vascular measurement.

17. The method of claim 16 wherein acquiring the peripheral vascular measurement comprises acquiring with a sensor connected with the ultrasound imaging system.

18. The method of claim 17 wherein acquiring with the sensor comprises acquiring with a tonometer connected with the ultrasound imaging system.

19. The method of claim 16 wherein determining with the processor comprises determining with a device in the ultrasound imaging system.

20. The method of claim 16 wherein determining the risk or diagnosis comprises characterizing operation of (1) atria or veins, (2) ventricle, and (3) arteries, arterioles or capillaries each as a function of the ultrasound data, the peripheral vascular measurement, or combinations thereof, at least one of (1), (2) or (3) being a function of the peripheral vascular measurement.

21. The method of claim 16 wherein determining the risk, data validity or diagnosis comprises comparing the peripheral vascular measurement and the ultrasound data to a knowledge base.

22. The method of claim 16 wherein acquiring the ultrasound data and acquiring the peripheral vascular measurement comprise acquiring both the ultrasound data and the peripheral vascular measurement in an automated workflow of a single user interface.

23. In a computer readable storage medium having stored therein data representing instructions executable by a programmed processor for medical diagnostic ultrasound, the storage medium comprising instructions for:
receiving ultrasound data and pressure data;
determining a risk, data validity or diagnosis as a function of both the ultrasound data and the peripheral vascular measurement; and
outputting the risk, data validity or diagnosis.

24. The instructions of claim 23 wherein the pressure data comprises a tonometer measurement.

25. The instructions of claim 23 wherein determining the risk, data validity or diagnosis comprises characterizing operation of (1) atria or veins and (2) ventricle as a function of the ultrasound data and characterizing operation of (3) arteries, arterioles or capillaries as a function of the pressure data, the pressure data being a central aortic pressure.

26. The instructions of claim 23 wherein determining the risk or diagnosis comprises comparing the peripheral vascular measurement and the ultrasound data to a knowledge base.

27. The instructions of claim 23 further comprising:
acquiring both the ultrasound data and the peripheral vascular measurement in an automated workflow of a single user interface.
